(19) United States
(12) Patent Application Publication
(10) Pub. No.: US 2010/0273185 A1
(43) Pub. Date: Oct. 28, 2010

(54) Detection of Biased Agonist Activation

(75) Inventors: Eric Sandberg, Dupont, WA (US); Michael Getman, Ashland, MA (US); Rafael Fernandez, Jamaica Plain, MA (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: SRU Biosystems, Inc.

(21) Appl. No.: 12/768,269

(22) Filed: Apr. 27, 2010

Related U.S. Application Data
(60) Provisional application No. 61/172,952, filed on Apr. 27, 2009.

(51) Int. Cl. G01N 33/543

(52) U.S. Cl. 435/7.2

(57) ABSTRACT

The invention provides single assay methods to detect the activity of biased agonists or agonists that are less than full agonists on cells.

### PRIORITY

This application claims the benefit of U.S. Ser. No. 61/172,952, which was filed on Apr. 27, 2010, which is incorporated by reference herein in its entirety.

### BACKGROUND OF THE INVENTION

Currently, multiple assays must be conducted to detect biased agonist activation. For instance, to demonstrate that carvedilol was a biased agonist, it was first shown that carvedilol does not activate G protein signaling, but does activate ERK1/2 in cells expressing the wild type β-2 adrenergic receptor. Then, carvedilol was shown to activate ERK1/2 in cells expressing a mutant β-2 adrenergic receptor that cannot couple to G proteins. This demonstrated that ERK1/2 activation by carvedilol occurred independently of G protein signaling. Finally, to show that ERK1/2 activation by carvedilol was due to β-arrestin activity, siRNA knockdown of β-arrestin was used. See Wisler et al., PNAS, 104:16657 (2007).

Single assay methods are needed to detect activity of a ligand or stimuli, such as a GPCR ligand, that preferentially activates one or more particular pathways (e.g., β-arrestin signal transduction) to the exclusion of other possible pathways. Quick and inexpensive methods of detecting biased agonists or agonists that are less than full agonists are needed in the art.

### SUMMARY OF THE INVENTION

One embodiment of the invention provides a method of determining if a ligand or stimulus is a biased agonist. The method comprises immobilizing or placing one or more cells on a surface of a colorimetric resonant reflectance optical biosensor and applying a ligand or stimulus to the one or more cells. Colorimetric resonant reflectance optical peak wavelength values (PWVs) are detected for the one or more cells over time. The PWVs are compared to PWVs over time of a full agonist acting on the same or similar one or more cells. The ligand or stimulus is a biased agonist if the PWVs of the ligand or stimulus over time are smaller in magnitude or later in onset or both than the PWVs over time of the full agonist. No detection labels can be associated with the cells, ligand and stimulus. The PWVs can be detected for five or more minutes. The one or more cells can be immobilized to the surface of the colorimetric resonant reflectance optical biosensor by one or more specific binding substances. The one or more specific binding substances can be one or more extracellular matrix ligands.

Still another embodiment of the invention provides a method of determining if a ligand or stimulus is a not a full agonist. The method comprises immobilizing or placing one or more cells on a surface of a colorimetric resonant reflectance optical biosensor and applying a ligand or stimulus to the one or more cells. The colorimetric resonant reflectance optical peak wavelength values (PWVs) are detected for the one or more cells over time. The PWVs are compared to PWVs over time of a full agonist acting on the same or similar one or more cells. The ligand or stimulus is not a full agonist if the PWVs of the ligand or stimulus over time are smaller in magnitude or later in onset or both than the PWVs over time of the full agonist.

Yet another embodiment of the invention provides a method of determining if a ligand or stimulus is a biased agonist. The method comprises immobilizing or placing one or more cells on a surface of a grating based waveguide biosensor and applying a ligand or stimulus to the one or more cells. Changes in effective refractive indices for the one or more cells are detected over time. The effective refractive indices are compared to effective refractive indices over time of a full agonist acting on the same or similar one or more cells. The ligand or stimulus is a biased agonist if the effective refractive indices of the ligand or stimulus over time are smaller in magnitude or later in onset or both than the effective refractive indices over time of the full agonist. No detection labels can be associated with the cells, ligand and stimulus. The effective refractive indices can be detected for five or more minutes. The one or more cells can be immobilized to the surface of the grating based waveguide biosensor by one or more specific binding substances. The one or more specific binding substances can be one or more extracellular matrix ligands.

Even another embodiment of the invention provides a method of determining if a ligand or stimulus is a not a full agonist. The method comprises immobilizing or placing one or more cells on a surface of a grating based waveguide biosensor and applying a ligand or stimulus to the one or more cells. Changes in effective refractive indices are detected for the one or more cells over time. The effective refractive indices are compared to effective refractive indices over time of a full agonist acting on the same or similar one or more cells.

### BRIEF DESCRIPTION OF THE DRAWINGS

FIGS. 1A-B shows the response of HEK293 cells expressing AT1R to Ang II, a full agonist, or SII, a biased agonist.

FIGS. 2A-B shows the response of HEK293 cells expressing β2AR to isoproterenol, a full agonist, or carvediolol, a biased agonist.

FIGS. 3A-B shows the real time PWV responses of HEK293 cells expressing β2AR to isoproterenol, a full agonist, or carvediolol, a biased agonist, and siRNA molecules specific for β-arrestin 1 and 2 proteins or control siRNA molecules.

FIGS. 4A-B show the maximal cell response of HEK293 cells expressing β2AR to isoproterenol, a full agonist or carvediolol, a biased agonist and siRNA molecules specific for β-arrestin 1 and 2 proteins or control siRNA molecules.

### DETAILED DESCRIPTION OF THE INVENTION

As used herein, the singular forms “a,” “an”, and “the” include plural referents unless the context clearly dictates otherwise.

### Biosensors

Biosensors of the invention can be colorimetric resonant reflectance biosensors. See e.g., Cunningham et al., “Colorimetric resonant reflection as a direct biochemical assay technique,” Sensors and Actuators B, Volume 81, p. 316-328, Jan. 5, 2002; U.S. Pat. Publ. No. 2004/0091397; U.S. Pat. No. 7,094,595; U.S. Pat. No. 7,264,973. Colorimetric resonant reflectance biosensors are not surface plasmon resonant (SPR) biosensors. SPR biosensors have a thin metal layer, such as silver, gold, copper, aluminum, sodium, and indium. The metal must have conduction band electrons capable of resonating with light at a suitable wavelength. A SPR biosensor surface exposed to light must be pure metal. Oxides, sulfides and other films interfere with SPR. Colorimetric resonant biosensors do not have a metal layer, rather they have a dielectric coating of high refractive index material, such as zinc sulfide, titanium dioxide, tantalum oxide, and silicon nitride.

Grating-based waveguide biosensors can be used in the methods of the invention. Grating-based waveguide biosensors are described in, e.g., U.S. Pat. No. 5,738,825. A grating-based waveguide biosensor comprises a waveguiding film and a diffraction grating that incouples an incident light field into the waveguiding film to generate a diffracted light field. A change in the effective refractive index of the waveguiding film is detected. Devices where the wave must be transported a significant distance within the device, such as grating-based waveguide biosensors, lack the spatial resolution of colorimetric resonant reflectance biosensors. Where grating-based waveguide biosensors are used instead of colorimetric resonant reflectance biosensors changes in effective refractive index are determined over time instead of changes or shifts in PWVs. Therefore, changes in effective refractive index can be substituted for changes or shifts in PWV throughout the specification.

A colorimetric resonant reflectance biosensor allows biochemical interactions to be measured on the biosensor's surface without the use of fluorescent tags, colorimetric labels or any other type of detection tag or detection label. A biosensor surface contains an optical structure that, when illuminated with collimated and/or white light, is designed to reflect only a narrow band of wavelengths (“a resonant grating effect”). The narrow wavelength band is described as a wavelength “peak.” The “peak wavelength value” (PWV) changes when materials, such as biological materials, are deposited or removed from the biosensor surface. PWV changes can also be detected when cells are activated by, e.g., an agonist. A readout instrument is used to illuminate distinct locations on a biosensor surface with collimated and/or white light, and to collect reflected light. The collected light is gathered into a wavelength spectrometer for determination of a PWV.

A biosensor can be incorporated into standard disposable laboratory items such as microtiter plates by bonding the structure (biosensor side up) into the bottom of a bottomless microtiter plate cartridge. Incorporation of a biosensor into common laboratory format cartridges is desirable for compatibility with existing microtiter plate handling equipment such as mixers, incubators, and liquid dispensing equipment. Biosensors can also be incorporated into, e.g., microfluidic, macrofluidic, or microarray devices (see, e.g., U.S. Pat. No. 7,033,819, U.S. Pat. No. 7,033,821). Biosensors can be used with well-know methodology in the art (see, e.g., Methods of Molecular Biology edited by Jun-Lin Guan, Vol. 294, Humana Press, Totowa, N.J.) to monitor cell changes or the lack of these changes upon exposure to one or more ligands or stimuli.

Colorimetric resonant reflectance biosensors comprise subwavelength structured surfaces (SWS) and are an unconventional type of diffractive optic that can mimic the effect of thin-film coatings. (Peng & Morris, “Resonant scattering from two-dimensional gratings,” J. Opt. Soc. Am. A, Vol. 13, No. 5, p. 993, May 1996; Magnusson, & Wang, “New principle for optical filters,” Appl. Phys. Lett., 61, No. 9, p. 1022, August, 1992; Peng & Morris, “Experimental demonstration of resonant anomalies in diffraction from two-dimensional gratings,” Optics Letters, Vol. 21, No. 8, p. 549, April, 1996). A SWS structure contains a one-dimensional, two-dimensional, or three dimensional grating in which the grating period is small compared to the wavelength of incident light so that no diffractive orders other than the reflected and transmitted zeroth orders are allowed to propagate. Propagation of guided modes in the lateral direction are not supported. Rather, the guided mode resonant effect occurs over a highly localized region of approximately 3 microns from the point that any photon enters the biosensor structure.

The reflected or transmitted light of a colorimetric resonant reflectance biosensor can be modulated by the addition of molecules such as specific binding substances or cells to the upper surface of the biosensor. The added molecules increase the optical path length of incident radiation through the structure, and thus modify the wavelength at which maximum reflectance or transmittance will occur.

In one embodiment, a colorimetric resonant reflectance biosensor, when illuminated with white and/or collimated light, is designed to reflect a single wavelength or a narrow band (about 1 to about 10 nm) of wavelengths (a “resonant grating effect”). When mass is deposited on the surface of the biosensor, the reflected wavelength is shifted due to the change of the optical path of light that is shown on the biosensor.

A detection system consists of, for example, a light source that illuminates a small spot of a biosensor at normal incidence through, for example, a fiber optic probe, and a spectrometer that collects the reflected light through, for example, a second fiber optic probe also at normal incidence. Because no physical contact occurs between the excitation/detection system and the biosensor surface, no special coupling prisms are required and the biosensor can be easily adapted to any commonly used assay platform including, for example, microtiter plates. A single spectrometer reading can be performed in several milliseconds, thus it is possible to quickly measure a large number of molecular interactions taking place in parallel upon a biosensor surface, and to monitor reaction kinetics in real time.

A colorimetric resonant reflectance biosensor comprises, e.g., an optical grating comprised of a high refractive index material, a substrate layer that supports the grating, and optionally one or more specific binding substances or linkers immobilized on the surface of the grating opposite of the substrate layer. The high refractive index material has a higher refractive index than a substrate layer. See, e.g., U.S. Pat. No. 7,094,595; U.S. Pat. No. 7,070,987. Optionally, a cover layer covers the grating surface. An optical grating is coated with a high refractive index dielectric film which can be comprised of a material that includes, for example, zinc sulfide, titanium dioxide, tantalum oxide, silicon nitride, and silicon dioxide. A cross-sectional profile of a grating with optical features can comprise any periodically repeating function, for example, a “square-wave.” An optical grating can also comprise a repeating pattern of shapes selected from the group consisting of lines (one-dimensional), squares, circles, ellipses, triangles, trapezoids, sinusoidal waves, ovals, rectangles, and hexagons. A colorimetric resonant reflectance biosensor of the invention can also comprise an optical grating comprised of, for example, plastic or epoxy, which is coated with a high refractive index material.

Linear gratings (i.e., one dimensional gratings) have resonant characteristics where the illuminating light polarization is oriented perpendicular to the grating period. A colorimetric resonant reflection biosensor can also comprise, for example, a two-dimensional grating, e.g., a hexagonal array of holes or squares. Other shapes can be used as well. A linear grating has the same pitch (i.e. distance between regions of high and low refractive index), period, layer thicknesses, and material properties as a hexagonal array grating. However, light must be polarized perpendicular to the grating lines in order to be resonantly coupled into the optical structure. Therefore, a polarizing filter oriented with its polarization axis perpendicular to the linear grating must be inserted between the illumination source and the biosensor surface. Because only a small portion of the illuminating light source is correctly polarized, a longer integration time is required to collect an equivalent amount of resonantly reflected light compared to a hexagonal grating.

An optical grating can also comprise, for example, a “stepped” profile, in which high refractive index regions of a single, fixed height are embedded within a lower refractive index cover layer. The alternating regions of high and low refractive index provide an optical waveguide parallel to the top surface of the biosensor.

A colorimetric resonant reflectance biosensor of the invention can further comprise a cover layer on the surface of an optical grating opposite of a substrate layer. Where a cover layer is present, the one or more specific binding substances or cells are immobilized on the surface of the cover layer opposite of the grating. Preferably, a cover layer comprises a material that has a lower refractive index than a material that comprises the grating. A cover layer can be comprised of, for example, glass (including spin-on glass (SOG)), epoxy, or plastic.

For example, various polymers that meet the refractive index requirement of a biosensor can be used for a cover layer. SOG can be used due to its favorable refractive index, ease of handling, and readiness of being activated with specific binding substances using the wealth of glass surface activation techniques. When the flatness of the biosensor surface is not an issue for a particular system setup, a grating structure of SiN/glass can directly be used as the sensing surface, the activation of which can be done using the same means as on a glass surface.

Resonant reflection can also be obtained without a planarizing cover layer over an optical grating. For example, a biosensor can contain only a substrate coated with a structured thin film layer of high refractive index material. Without the use of a planarizing cover layer, the surrounding medium (such as air or water) fills the grating. Therefore, specific binding substances are immobilized to the biosensor on all surfaces of an optical grating exposed to the specific binding substances, rather than only on an upper surface.

In general, a colorimetric resonant reflectance biosensor can be illuminated with white and/or collimated light that will contain light of every polarization angle. The orientation of the polarization angle with respect to repeating features in a biosensor grating will determine the resonance wavelength. For example, a “linear grating” (i.e., a one-dimensional grating) biosensor consisting of a set of repeating lines and spaces will have two optical polarizations that can generate separate resonant reflections. Light that is polarized perpendicularly to the lines is called “s-polarized,” while light that is polarized parallel to the lines is called “p-polarized.” Both the s and p components of incident light exist simultaneously in an unfiltered illumination beam, and each generates a separate resonant signal. A biosensor can generally be designed to optimize the properties of only one polarization (the s-polarization), and the non-optimized polarization is easily removed by a polarizing filter.

In order to remove the polarization dependence, so that every polarization angle generates the same resonant reflection spectra, an alternate biosensor structure can be used that consists of a set of concentric rings. In this structure, the difference between the inside diameter and the outside diameter of each concentric ring is equal to about one-half of a grating period. Each successive ring has an inside diameter that is about one grating period greater than the inside diameter of the previous ring. The concentric ring pattern extends to cover a single sensor location—such as an array spot or a microtiter plate well. Each separate microarray spot or microtiter plate well has a separate concentric ring pattern centered within it. All polarization directions of such a structure have the same cross-sectional profile. The concentric ring structure must be illuminated precisely on-center to preserve polarization independence. The grating period of a concentric ring structure is less than the wavelength of the resonantly reflected light. The grating period is about 0.01 micron to about 1 micron. The grating depth is about 0.01 to about 1 micron.

In another embodiment, an array of holes or posts are arranged to closely approximate the concentric circle structure described above without requiring the illumination beam to be centered upon any particular location of the grid. Such an array pattern is automatically generated by the optical interference of three laser beams incident on a surface from three directions at equal angles. In this pattern, the holes (or posts) are centered upon the corners of an array of closely packed hexagons. The holes or posts also occur in the center of each hexagon. Such a hexagonal grid of holes or posts has three polarization directions that “see” the same cross-sectional profile. The hexagonal grid structure, therefore, provides equivalent resonant reflection spectra using light of any polarization angle. Thus, no polarizing filter is required to remove unwanted reflected signal components. The period of the holes or posts can be about 0.01 microns to about 1 micron and the depth or height can be about 0.01 microns to about 1 micron.

A detection system can comprise a colorimetric resonant reflectance biosensor a light source that directs light to the colorimetric resonant reflectance biosensor, and a detector that detects light reflected from the biosensor. In one embodiment, it is possible to simplify the readout instrumentation by the application of a filter so that only positive results over a determined threshold trigger a detection.

By measuring the shift in resonant wavelength at each distinct location of a colorimetric resonant reflectance biosensor of the invention, it is possible to determine which distinct locations have, e.g., biological material deposited on them, or interactions occurring between ligands or stimuli and cells at the distinct locations. The extent of the shift can be used to determine, e.g., the amount of binding partners in a test sample, the chemical affinity between one or more specific binding substances and the binding partners of the test sample, and/or changes in cells due to exposure to ligands or stimuli.

A colorimetric resonant reflectance biosensor can be illuminated two or more times. A first measurement can determine the reflectance spectra of one or more distinct locations of a biosensor, e.g., before cells are added to the biosensor. A second measurement can determine the reflectance spectra after, e.g., one or more cells are applied to a biosensor. A third measurement can determine the reflectance spectra after ligands and/or stimuli are added to the cells. The difference in peak wavelength between the measurements is a measurement of the presence, amount, or status of cells on the biosensor. This method of illumination can control for small imperfections in a surface of a biosensor that can result in regions with slight variations in the peak resonant wavelength. This method can also control for varying concentrations or density of cell matter on a biosensor. Alternatively, measurements (PWVs) can be taken in real time over the course of an assay (e.g., about every 1, 5, 10, 20, 30, 40, 50, 60 seconds, or every 1, 2, 5, 10, 20, 30, 40, 50, or 60 minutes (or any range between about 1 second and 60 minutes) during the time course of an assay. A time course can begin prior to the cells being added to the biosensor and can end about 1, 2, 3, 4, 5, 10, 20, 30, 40, 50, or 60 minutes or about 1, 2, 3, 4, 5, 10, 20, 24, 36, 48 or more hours after ligands or stimuli are added to the biosensor surface. Alternatively, a time course can start when cells are added to the biosensor surface or before ligands are added to the biosensor surface or when ligands are added to the biosensor surface and can end about 1, 2, 3, 4, 5, 10, 20, 30, 40, 50, or 60 minutes or about 1, 2, 3, 4, 5, 10, 20, 24, 36, 48 or more hours after ligands or stimuli are added to the biosensor surface.

### Surface of Biosensor

Immobilization of one or more specific binding substances onto a biosensor is performed so that a specific binding substance will not be washed away by any rinsing procedures, and so that the binding of the specific binding substance to binding partners (e.g., cells) in a test sample is unimpeded by the biosensor surface. One or more specific binding substances can be attached to a biosensor surface by physical adsorption (i.e., without the use of chemical linkers) or by chemical binding (i.e., with the use of chemical linkers) as well as electrochemical binding, electrostatic binding, hydrophobic binding and hydrophilic binding. Chemical binding can generate stronger attachment of specific binding substances on a biosensor surface and provide defined orientation and conformation of the surface-bound molecules.

Furthermore, specific binding substances can be arranged in an array of one or more distinct locations on the biosensor surface. The biosensor surface can reside within one or more wells of a multiwell plate and comprising one or more surfaces of the multiwell plate or microarray. The array of specific binding substances comprises one or more specific binding substances on the biosensor surface or within a microwell plate with a biosensor surface, such that a surface contains one or more distinct locations, each with a different specific binding substance or population of cells. For example, an array can comprise 1, 2, 3, 5, 10, 100, 1,000, 10,000 or 100,000 or greater distinct locations. Thus, each well of a multiwell plate or microarray can have within it an array of one or more distinct locations separate from the other wells of the multiwell plate, which allows multiple different samples to be processed on one multiwell plate. The array or arrays within any one well can be the same or different than the array or arrays found in any other microtiter wells of the same microtiter plate.

Immobilization of a specific binding substance to a biosensor surface can be also be affected via binding to, for example, the following functional linkers: a nickel group, an amine group, an aldehyde group, an acid group, an alkane group, an alkene group, an alkyne group, an aromatic group, an alcohol group, an ether group, a ketone group, an ester group, an amide group, an amino acid group, a nitro group, a nitrile group, a carbohydrate group, a thiol group, an organic phosphate group, a lipid group, a phospholipid group or a steroid group. Furthermore, a specific binding substance can be immobilized on the surface of a biosensor via physical adsorption, chemical binding, electrochemical binding, electrostatic binding, hydrophobic binding or hydrophilic binding, and immunocapture methods.

In one embodiment of the invention a biosensor can be coated with a linker such as, e.g., a nickel group, an amine group, an aldehyde group, an acid group, an alkane group, an alkene group, an alkyne group, an aromatic group, an alcohol group, an ether group, a ketone group, an ester group, an amide group, an amino acid group, a nitro group, a nitrile group, a carbohydrate group, a thiol group, an organic phosphate group, a lipid group, a phospholipid group or a steroid group. For example, an amine surface can be used to attach several types of linker molecules while an aldehyde surface can be used to bind proteins directly, without an additional linker. A nickel surface can be used to bind molecules that have an incorporated histidine (“his”) tag. Detection of “his-tagged” molecules with a nickel-activated surface is well known in the art (Whitesides, Anal. Chem. 68, 490, (1996)).

Linkers and specific binding substances can be immobilized on the surface of a biosensor such that each well has the same linker and/or specific binding substances immobilized therein. Alternatively, each well can contain a different combination of linkers and/or specific binding substances.

Specific binding substances can specifically or non-specifically bind to a linker immobilized on the surface of a biosensor. Alternatively, the surface of the biosensor can have no linker and a specific binding substance or population of cells can bind to the biosensor surface non-specifically.

Immobilization of one or more specific binding substances onto a biosensor is performed so that a specific binding substance will not be washed away by rinsing procedures, and so that its binding to binding partner (e.g. a cell) is unimpeded by the biosensor surface. Several different types of surface chemistry strategies have been implemented for covalent attachment of specific binding substances to, for example, glass for use in various types of microarrays and biosensors. These same methods can be readily adapted to a biosensor of the invention.

One or more specific binding substances can be attached to a biosensor surface by physical adsorption (i.e., without the use of chemical linkers) or by chemical binding (i.e., with the use of chemical linkers) as well as electrochemical binding, electrostatic binding, hydrophobic binding and hydrophilic binding. Chemical binding can generate stronger attachment of specific binding substances on a biosensor surface and provide defined orientation and conformation of the surface-bound molecules.

Immobilization of specific binding substances to plastic, epoxy, or high refractive index material can be performed essentially as described for immobilization to glass. However, the acid wash step can be eliminated where such a treatment would damage the material to which the specific binding substances are immobilized.

### Specific Binding Substances

A specific binding substance is a molecule that binds to another molecule on or within a cell. A specific binding substance binds to a cell such that the cell becomes immobilized to the surface of a biosensor. A specific binding substance can be, for example, a nucleic acid, peptide, extracellular matrix (ECM) ligand, protein solution, peptide solution, single or double stranded DNA solution, RNA solution, RNA-DNA hybrid solution, a solution containing compounds from a combinatorial chemical library, antigen, polyclonal antibody, monoclonal antibody, single chain antibody (scFv), F(ab) fragment, F(ab′)₂ fragment, Fv fragment, small organic molecule, cell, virus, bacteria, or polymer. The polymer is selected from the group of long chain molecules with multiple active sites per molecule consisting of hydrogel, dextran, poly-amino acids and derivatives thereof, including poly-lysine (comprising poly-l-lysine and poly-d-lysine), poly-phe-lysine and poly-glu-lysine.

Integrins are cell surface receptors that interact with the extracellular matrix (ECM) and mediate intracellular signals. Integrins are responsible for cytoskeletal organization, cellular motility, regulation of the cell cycle, regulation of cellular of integrin affinity, attachment of cells to viruses, attachment of cells to other cells or ECM. Integrins are also responsible for signal transduction, a process whereby the cell transforms one kind of signal or stimulus into another intracellularly and intercellularly. Integrins can transduce information from the ECM to the cell and information from the cell to other cells (e.g., via integrins on the other cells) or to the ECM. A list of integrins and their ECM ligands can be found in Takada et al., Genome Biology 8:215 (2007). ECM ligands can be used as specific binding substances in the methods of the invention.

Other cell surface receptors that interact with the ECM include focal adhesion proteins. Focal adhesion proteins form large complexes that connect the cytoskeleton of a cell to the ECM. Focal adhesion proteins include, for example, talin, α-actinin, filamin, vinculin, focal adhesion kinase, paxilin, parvin, actopaxin, nexilin, fimbrin, G-actin, vimentin, syntenin, and many others.

Yet other cell surface receptors can include, but are not limited to those that can interact with the ECM include cluster of differentiation (CD) molecules. CD molecules act in a variety of ways and often act as receptors or ligands for the cell. Other cell surface receptors that interact with ECM include cadherins, adherins, and selectins.

One embodiment of the invention provides for the use of colorimetric resonant reflection biosensor technology or other biosensor technology, including for example, grating based waveguide biosensor technology, to measure or detect the interaction of cells with ligands or stimuli.

In one embodiment, by specifically immobilizing cells to a biosensor surface through binding of cell surface receptors, such as integrins, to ECM ligands that are immobilized to the biosensor, the binding of the cells to the biosensor and the cells' response to ligands and/or stimuli is dramatically altered as compared to cells that are non-specifically immobilized to a biosensor surface. That is, detection of response of cells to stimuli and/or ligands is greatly enhanced or augmented where cells are immobilized to a biosensor via ECM ligand binding. See, U.S. Ser. No. 12/335,393, (US 2009/0130707) filed Dec. 15, 2008. In another embodiment of the invention, the cells are in a serum-free medium. A serum-free medium contains about 10, 5, 4, 3, 2, 1, 0.5% or less serum. See id. A serum-free medium can comprise about 0% serum or about 0% to about 1% serum.

In one embodiment of the invention, an ECM ligand is purified. A purified ECM ligand is an ECM ligand preparation that is substantially free of cellular material, other types of ECM ligands, chemical precursors, chemicals used in preparation of the ECM ligand, or combinations thereof. An ECM ligand preparation that is substantially free of other types of ECM ligands, cellular material, culture medium, chemical precursors, chemicals used in preparation of the ECM ligand, etc., has less than about 30%, 20%, 10%, 5%, 1% or more of other ECM ligands, culture medium, chemical precursors, and/or other chemicals used in preparation. Therefore, a purified ECM ligand is about 70%, 80%, 90%, 95%, 99% or more pure. A purified ECM ligand does not include unpurified or semi-purified preparations or mixtures of ECM ligands that are less than 70% pure, e.g., fetal bovine serum. In one embodiment of the invention, ECM ligands are not purified and comprise a mixture of ECM proteins and non-ECM proteins. Examples of non-purified ECM ligand preparations include fetal bovine serum, bovine serum albumin, and ovalbumin.

In one embodiment of the invention detection of cell binding to ECM ligands is increased by about 2, 3, 4, 5, 6, 7, 8, 9, 10, 15, 20 or more times (or any range between 2 and 20 times) when the ECM ligand is specific for a cell surface receptor, e.g., an integrin or focal adhesion protein, present on the surface of the cells. In another embodiment of the invention detection of cellular responses to stimuli is increased by about 2, 3, 4, 5, 6, 7, 8, 9, 10, 15, 20 or more times (or any range between 2 and 20 times) when the cell is immobilized to the biosensor surface by an ECM ligand that is specific for a cell surface receptor, e.g., an integrin. “Specifically binds,” “specifically bind” or “specific for” means that a cell surface receptor, e.g., an integrin or focal adhesion protein, binds to a cognate extracellular matrix ligand, with greater affinity than to other, non-specific molecules. A non-specific molecule does not substantially bind to the cell receptor. For example, the integrin α4/β1 specifically binds the ECM ligand fibronectin, but does not specifically bind the non-specific ECM ligands collagen or laminin.

An array on a biosensor of the invention can be created by placing microdroplets of one or more specific binding substances onto, for example, an x-y grid of locations on a biosensor surface. When the biosensor is exposed to cells having receptors for specific binding substances, the cells will be preferentially attracted to distinct locations on the microarray that comprise specific binding substances that have high affinity for the cells. Some of the distinct locations will gather cells onto their surface, while other locations may not.

A specific binding substance specifically binds to a cell or molecule on the cell that is added to the surface of a biosensor of the invention such that the cell becomes immobilized to the biosensor. A specific binding substance specifically binds to its cognate receptor on a cell, but does not substantially bind other cell receptors. For example, where the specific binding substance is an antibody and its cellular receptor is a particular antigen, the antibody specifically binds to the particular antigen of the cell surface, but does not substantially bind other antigens. In one embodiment of the invention a cell receptor can bind a specific binding substance immobilized on the biosensor, wherein the receptor is on the surface of a cell.

While microtiter plates are the most common format used for biochemical assays, microarrays are increasingly seen as a means for maximizing the number of biochemical interactions that can be measured at one time while minimizing the volume of precious reagents. By application of specific binding substances with a microarray spotter onto a biosensor of the invention, specific binding substance densities of 10,000 specific binding substances/in² can be obtained. By focusing an illumination beam to interrogate a single microarray location, a biosensor can be used as a label-free microarray readout system.

### Liquid-Containing Vessels

A biosensor of the invention can comprise an inner surface, for example, a bottom surface of a liquid-containing vessel. A liquid-containing vessel can be, for example, a microtiter plate well, a test tube, a petri dish, or a microfluidic channel. One embodiment of this invention is a biosensor that is incorporated into any type of microtiter plate. For example, a biosensor can be incorporated into the bottom surface of a microtiter plate by assembling the walls of the reaction vessels over the biosensor surface, so that each reaction “spot” can be exposed to a distinct test sample. Therefore, each individual microtiter plate well can act as a separate reaction vessel. Separate chemical reactions can, therefore, occur within adjacent wells without intermixing reaction fluids and chemically distinct test solutions can be applied to individual wells.

Several methods for attaching a biosensor or grating of the invention to the bottom surface of bottomless microtiter plates can be used, including, for example, adhesive attachment, ultrasonic welding, and laser welding.

The most common assay formats for pharmaceutical high-throughput screening laboratories, molecular biology research laboratories, and diagnostic assay laboratories are microtiter plates. The plates are standard-sized plastic cartridges that can contain about 2, 6, 8, 24, 48, 96, 384, 1536, 3456, 9600 or more individual reaction vessels arranged in a grid. Due to the standard mechanical configuration of these plates, liquid dispensing, robotic plate handling, and detection systems are designed to work with this common format. A biosensor of the invention can be incorporated into the bottom surface of a standard microtiter plate. Because the biosensor surface can be fabricated in large areas, and because the readout system does not make physical contact with the biosensor surface, an arbitrary number of individual biosensor areas can be defined that are only limited by the focus resolution of the illumination optics and the x-y stage that scans the illumination/detection probe across the biosensor surface.

### Detection Systems

A detection system can comprise a biosensor a light source that directs light to the biosensor, and a detector that detects light reflected from the biosensor. In one embodiment, it is possible to simplify the readout instrumentation by the application of a filter so that only positive results over a determined threshold trigger a detection.

A light source can illuminate a colorimetric resonant reflectance biosensor from its top surface, i.e., the surface to which one or more cells are immobilized or present or from its bottom surface. By measuring the shift in resonant wavelength at each distinct location of a biosensor of the invention, it is possible to determine which distinct locations have binding partners bound to them. The extent of the shift can be used to determine changes in cellular response due to the activation by agonists.

A biosensor can be illuminated twice. The first measurement determines the reflectance spectra of one or more distinct locations of a biosensor array with one or more cells or one or more types of cells immobilized or placed on the biosensor. The second measurement determines the reflectance spectra after one or more ligands or stimuli are applied to a biosensor. The difference in peak wavelength between these two measurements is a measurement of the response of the cells to one or more ligands or stimuli a one or more distinct locations or wells of a biosensor. This method of illumination can control for small nonuniformities in a surface of a biosensor that can result in regions with slight variations in the peak resonant wavelength. This method can also control for varying concentrations or molecular weights of specific binding substances immobilized on a biosensor.

One type of detection system for illuminating the biosensor surface and for collecting the reflected light is a probe containing, for example, six illuminating optical fibers that are connected to a light source, and a single collecting optical fiber connected to a spectrometer. The number of fibers is not critical, any number of illuminating or collecting fibers are possible. The fibers are arranged in a bundle so that the collecting fiber is in the center of the bundle, and is surrounded by the six illuminating fibers. The tip of the fiber bundle is connected to a collimating lens that focuses the illumination onto the surface of the biosensor.

In this probe arrangement, the illuminating and collecting fibers are side-by-side. Therefore, when the collimating lens is correctly adjusted to focus light onto the biosensor surface, one observes six clearly defined circular regions of illumination, and a central dark region. Because the biosensor does not scatter light, but rather reflects a collimated beam, no light is incident upon the collecting fiber, and no resonant signal is observed. Only by defocusing the collimating lens until the six illumination regions overlap into the central region is any light reflected into the collecting fiber. Because only defocused, slightly uncollimated light can produce a signal, the biosensor is not illuminated with a single angle of incidence, but with a range of incident angles. The range of incident angles results in a mixture of resonant wavelengths. Thus, wider resonant peaks are measured than might otherwise be possible.

Therefore, it is desirable for the illuminating and collecting fiber probes to spatially share the same optical path. Several methods can be used to co-locate the illuminating and collecting optical paths. For example, a single illuminating fiber, which is connected at its first end to a light source that directs light at the biosensor, and a single collecting fiber, which is connected at its first end to a detector that detects light reflected from the biosensor, can each be connected at their second ends to a third fiber probe that can act as both an illuminator and a collector. The third fiber probe is oriented at a normal angle of incidence to the biosensor and supports counter-propagating illuminating and reflecting optical signals.

Another method of detection involves the use of a beam splitter that enables a single illuminating fiber, which is connected to a light source, to be oriented at a 90 degree angle to a collecting fiber, which is connected to a detector. Light is directed through the illuminating fiber probe into the beam splitter, which directs light at the biosensor. The reflected light is directed back into the beam splitter, which directs light into the collecting fiber probe. A beam splitter allows the illuminating light and the reflected light to share a common optical path between the beam splitter and the biosensor, so perfectly collimated light can be used without defocusing.

### Angular Scanning

Detection systems of the invention are based on collimated white light illumination of a biosensor surface and optical spectroscopy measurement of the resonance peak of the reflected beam. Molecular binding on the surface of a biosensor is indicated by a shift in the peak wavelength value, while an increase in the wavelength corresponds to an increase in molecular absorption.

As shown in theoretical modeling and experimental data, the resonance peak wavelength is strongly dependent on the incident angle of the detection light beam. Because of the angular dependence of the resonance peak wavelength, the incident white light needs to be well collimated. Angular dispersion of the light beam broadens the resonance peak, and reduces biosensor detection sensitivity. In addition, the signal quality from the spectroscopic measurement depends on the power of the light source and the sensitivity of the detector. In order to obtain a high signal-to-noise ratio, an excessively long integration time for each detection location can be required, thus lengthening overall time to readout a biosensor plate. A tunable laser source can be used for detection of grating resonance, but is expensive.

In one embodiment of the invention, these disadvantages are addressed by using a laser beam for illumination of a biosensor, and a light detector for measurement of reflected beam power. A scanning mirror device can be used for varying the incident angle of the laser beam, and an optical system is used for maintaining collimation of the incident laser beam. See, e.g., “Optical Scanning” (Gerald F. Marchall ed., Marcel Dekker (1991). Any type of laser scanning can be used. For example, a scanning device that can generate scan lines at a rate of about 2 lines to about 1,000 lines per second is useful in the invention. In one embodiment of the invention, a scanning device scans from about 50 lines to about 300 lines per second.

In one embodiment, the reflected light beam passes through part of the laser scanning optical system, and is measured by a single light detector. The laser source can be a diode laser with a wavelength of, for example, 780 nm, 785 nm, 810 nm, or 830 nm. Laser diodes such as these are readily available at power levels up to 150 mW, and their wavelengths correspond to high sensitivity of Si photodiodes. The detector thus can be based on photodiode biosensors. A light source provides light to a scanner device, which directs the light into an optical system. The optical system directs light to a biosensor. Light is reflected from the biosensor to the optical system, which then directs the light into a light signal detector. In one embodiment as the scanning mirror changes its angular position, the incident angle of the laser beam on the surface changes by nominally twice the mirror angular displacement. The scanning mirror device can be a linear galvanometer, operating at a frequency of about 2 Hz up to about 120 Hz, and mechanical scan angle of about 10 degrees to about 20 degrees. In this example, a single scan can be completed within about 10 msec. A resonant galvanometer or a polygon scanner can also be used. A simple optical system for angular scanning can consist of a pair of lenses with a common focal point between them. The optical system can be designed to achieve optimized performance for laser collimation and collection of reflected light beam.

The angular resolution depends on the galvanometer specification, and reflected light sampling frequency. Assuming galvanometer resolution of 30 arcsec mechanical, corresponding resolution for biosensor angular scan is 60 arcsec, i.e. 0.017 degree. In addition, assume a sampling rate of 100 ksamples/sec, and 20 degrees scan within 10 msec. As a result, the quantization step is 20 degrees for 1000 samples, i.e. 0.02 degree per sample. In this example, a resonance peak width of 0.2 degree, as shown by Peng and Morris (Experimental demonstration of resonant anomalies in diffraction from two-dimensional gratings, Optics Lett., 21:549 (1996)), will be covered by 10 data points, each of which corresponds to resolution of the detection system.

The advantages of such a detection system includes: excellent collimation of incident light by a laser beam, high signal-to-noise ratio due to high beam power of a laser diode, low cost due to a single element light detector instead of a spectrometer, and high resolution of resonance peak due to angular scanning.

### Methods of Using Biosensors

An “agonist” is a ligand that can bind to a receptor and initiate a cellular response. “Potency” is the amount of the ligand or stimulus that is required to produce a specified effect. Potency is dependant upon receptor density, efficiency of stimulus response, affinity and efficacy. “Affinity” is the propensity of a ligand to associate with a receptor (expressed as K_{D}). “Efficacy” is the extent of functional change imparted to a receptor. “Intrinsic activity” is the property of attraction between a ligand (or stimulus) and a receptor. A “full agonist” has affinity for and activates a receptor. A full agonist has full efficacy at that receptor. A “partial agonist” has affinity for and activates a receptor; however, partial agonists have only partial efficacy at the receptor relative to a full agonist.

A ligand can be an agonist. A ligand can be, for example, a nucleic acid, peptide, extracellular matrix (ECM) ligand, protein solution, peptide solution, single or double stranded DNA solution, RNA solution, RNA-DNA hybrid solution, a solution containing compounds from a combinatorial chemical library, antigen, polyclonal antibody, monoclonal antibody, single chain antibody (scFv), F(ab) fragment, F(ab′)₂ fragment, Fv fragment, small organic molecule, cell, virus, bacteria, or polymer. The polymer is selected from the group of long chain molecules with multiple active sites per molecule consisting of hydrogel, dextran, poly-amino acids and derivatives thereof, including poly-lysine (comprising poly-l-lysine and poly-d-lysine), poly-phe-lysine and poly-glu-lysine.

“Biased agonism” (also termed “functional selectivity,” “ligand induced differential signaling,” “agonist directed trafficking of receptor stimulus,” “protean agonism,” or “differential engagement”) is the ligand-dependant selectivity for signal transduction pathways in one receptor. Urban et al., J. Pharm. Exp. Therapeutics, 320:1 (2007). The pathway that is activated and the extent to which the pathway is activated can depend on the particular ligand that binds to a receptor or stimuli. Biased agonists can differentially activate only a subset of functions of a single receptor. Biased agonism can be expressed as differences in intrinsic activity or potency, but the two are interrelated. Id.

Examples of biased agonists include, for example, atypical agonists of 5-HT₂ serotonin receptors, agonist-selective regulation of μ-opoid and β-adrenergic receptors, biased agonism of D_{2L} and D₁ dopamine receptors, among others. See, Siehler, Biotechnol. J. 3:471 (2008); Lefkowitcz, Acta Physiol, 190:9 (2007). The identification and characterization of biased agonists is important because receptors can have different pharmacological profiles depending on the activating ligand or stimuli and other molecules that are present or absent (such as G proteins). See Gilchrist, TRENDS Pharmacol. Sci., 28: 431 (2007). Ligands and/or stimuli can be biased agonists

The invention provides a single assay method of using label-free biosensors to detect activity of a ligand or stimuli, such as a GPCR ligand, that preferentially activates one or more particular pathways (e.g., β-arrestin signal transduction) to the exclusion of other possible pathways. Biased agonists can be distinguished from full agonists by a different signature response when their activity on cells is monitored over time.

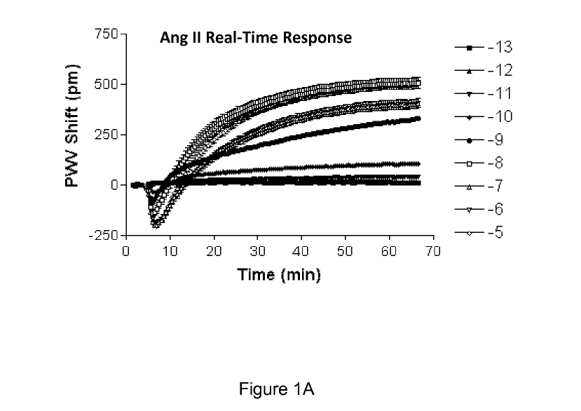

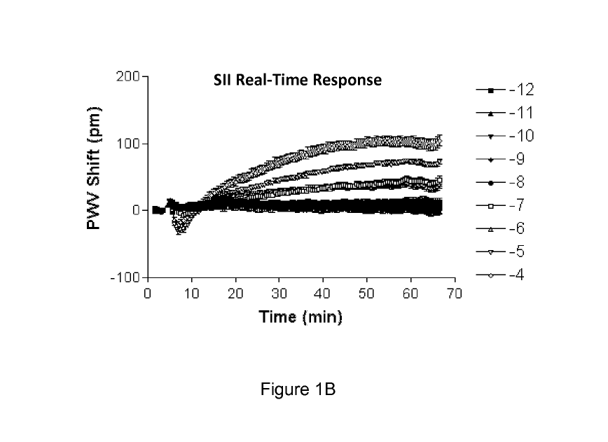

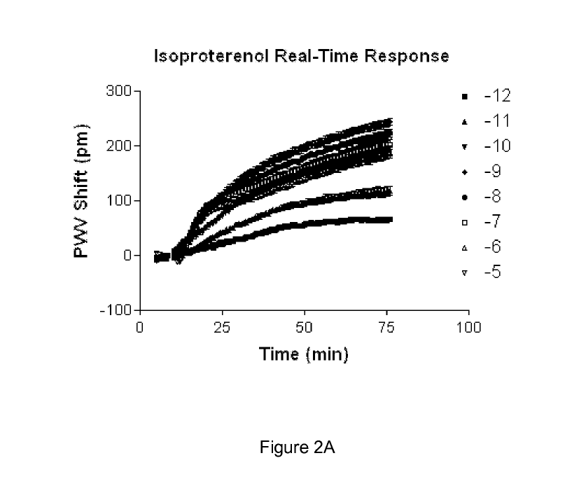

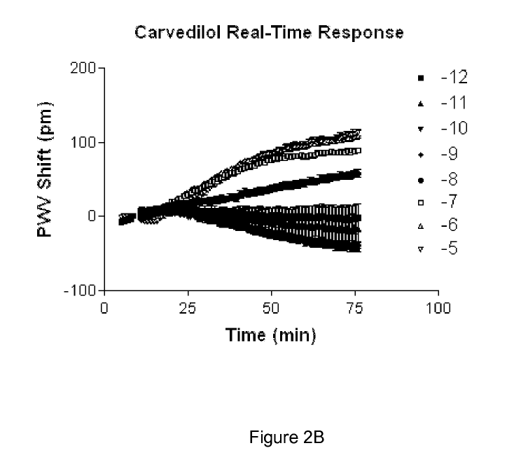

One embodiment of the invention provides a single assay method for detecting activation transduction pathways (e.g., of β-arrestin signaling) by biased agonists. Biased agonists or partial agonists can be distinguished from full agonists because the signals they generate are (1) smaller in magnitude and/or (2) later in onset. See FIGS. 1 and 2 (a CA2 BIND™ surface has ECMs present on the surface). For example, a biased agonist or partial agonist may generate PWV signals that are about 5, 10, 20, 30, 40, 50, 60, 70, 80, 90% or more (or any range between about 5 and 90%) less than PWV signals of a full agonist over a time course. A biased agonist or partial agonist may generate PWV signals that are about 5, 10, 20, 30, 40, 50, 60, 70, 80, 90% or more (or any range between about 5 and 90%) later in onset than that of a full agonist. In one embodiment of the invention, A biased agonist or partial agonist may generate PWV signals that haven an onset of about 5, 10, 30, 45 seconds, or 1, 2, 3, 4, 5, 6, 7, 8, 9, 10, 15, 20, 30, 60 or more minutes (or any range between about 5 seconds and 60 minutes) later in onset than that of a full agonist. Later in onset means that the cellular response (a positive PWV shift) occurs later in time than the cellular response of a full agonist.

Biosensors of the invention can be used to study one or a number of cell receptor/ligand or stimulus interactions in parallel. A stimulus can be, e.g., a change in temperature, pH, ion concentration, anion concentration, cation concentration, pressure, light, electromagnetic field, and/or magnetic field. Binding of one or more ligands to cell receptors or the effect of a stimulus on a cell receptor can be detected, without the use of labels, by applying cells having receptors to a biosensor that has one or more specific binding substances (such as extracellular matrix proteins, antigens, antibodies or other substance to which a cell will bind) immobilized on its surface. The one or more specific binding substances can bind a cell receptor or another component of a cell. Alternatively, no molecules or no molecules that bind to cells are present on the surface of the biosensor and cells are merely added to the surface and tested immediately or after an incubation of the cells on the surface. One of more types of cells (e.g., about 1, 2, 3, 4, 5, 10, 20, 30 or more types of cells) can be added to each distinct location or well. In one embodiment of the invention, one or more specific binding substances are one or more extracellular matrix protein ligands that can bind to receptors on cells, wherein the receptors (e.g., an integrin) are specific for extracellular matrix protein ligands. A biosensor can be illuminated with light and a maxima in reflected wavelength, or a minima in transmitted wavelength of light is detected from the biosensor. A change in effective refractive index can be detected from a grating based waveguide biosensors.

A ligand or stimulus is then added to the cells and the response of the cells to the ligand or stimulus is detected and can be compared to the response of the same or similar cells to known full agonists, partial agonists, or biased agonists. Where the ligand or stimulus has an effect on the cells the reflected wavelength of light is shifted as compared to a situation where the ligand or stimulus has no effect on the cells. Signals are detected from a grating-based waveguide biosensor and are compared to each other or to controls in a manner similar to that for colorimetric resonant reflectance biosensors. All assays or methods described herein can be performed on colorimetric resonant reflectance biosensors and grating-based waveguide biosensor.

One embodiment of the invention provides a method of determining if a ligand or stimulus is a biased agonist. The method comprises immobilizing or placing one or more cells on a surface of a colorimetric resonant reflectance optical biosensor and applying a ligand or stimulus to the one or more cells. Colorimetric resonant reflectance optical peak wavelength values (PWVs) are detected for the one or more cells over time. The PWVs are compared to PWVs over time of a full agonist acting on the same or similar one or more cells. The ligand or stimulus is a biased agonist if the PWVs of the ligand or stimulus over time are smaller in magnitude or later in onset or both than the PWVs over time of the full agonist. In one embodiment of the invention, no detection labels are associated with the cells, ligand or stimulus.

One embodiment of the invention provides a method of determining if a ligand or stimulus is a not a full agonist. The method comprises immobilizing or placing one or more cells on a surface of a colorimetric resonant reflectance optical biosensor and applying a ligand or stimulus to the one or more cells. Colorimetric resonant reflectance optical peak wavelength values (PWVs) are detected for the one or more cells over time. The PWVs are compared to PWVs over time of a full agonist acting on the same or similar one or more cells. The ligand or stimulus is not a full agonist if the PWVs of the ligand or stimulus over time are smaller in magnitude or later in onset or both than the PWVs over time of the full agonist. In one embodiment of the invention, no detection labels are associated with the cells, ligand or stimulus.

In one embodiment of the invention, a variety of specific binding substances, for example, ECM ligands or antibodies specific for cell receptors or cell antigens, can be immobilized in an array format onto a biosensor of the invention. The biosensor is then contacted with cells bearing ECM ligand receptors, e.g., integrins or focal adhesion proteins or other types of receptors. Only the cells that specifically bind to the specific binding substances are immobilized on the biosensor. It is important to note that cell receptors can be used to immobilize cells to a biosensor via, e.g., antibodies or ECM. Cell receptors can also be acted upon by ligands and/or stimuli during an assay. The cell receptors that may be used to immobilize the cell to the biosensor surface can be the same or different from the cell receptors that are acted upon by ligands and/or stimuli during an assay.

The use of an enzyme, fluorescent, or other detectable label is not required. For high-throughput applications, biosensors can be arranged in an array of arrays, wherein several biosensors comprising an array of specific binding substances are arranged in an array. Such an array of arrays can be, for example, dipped into microtiter plate to perform many assays at one time. In another embodiment, a biosensor can occur on the tip of a fiber probe for in vivo detection of biochemical substance. Each location in the array can have a different ligand or stimuli or combination of ligands or stimuli or different concentrations or amounts of ligands or stimuli. Different types of cells can be added to each location in the array.

One embodiment of the invention allows the direct detection of cell response to ligands or stimuli, as they occur in real time in response to ligands or stimuli with a colorimetric resonant reflectance biosensor or grating based waveguide biosensor and without the need to incorporate or without interference from radiometric, colorimetric, or fluorescent labels (although labels may be used if desired). Changes in cellular response to agonists can be detected as the cell is perturbed with ligands or stimuli. The cellular changes can then be detected in real time using a high speed, high resolution instrument, such as the BIND Scanner™ (i.e., a colorimetric resonant reflectance biosensor system), and corresponding algorithms to quantify data. See, e.g., U.S. Pat. No. 6,951,715 and U.S. Pat. Publ. 2004/0151626. By combining this methodology, instrumentation and computational analysis, cellular behavior can be expediently monitored in real time (i.e., expediently and conveniently observing and quantifying cell reactions during the instant the cell is responding to stimulus or ligand and over time while the cell is responding to the stimulus), in a label free manner.

Colorimetric resonant reflectance biosensors, such as SRU Biosystems, Inc. BIND™ technology (Woburn, Mass.) have the capability of measuring changes to a surface with respect to mass attachment from nanoscale biological systems. A BIND Scanner™ (i.e., a colorimetric resonant reflectance biosensor system) has a high resolution lens. The lens has a resolution of about 2-5, 2-10, 2-15, 2-20, 2-50, 2-100, 2-200 or 2-300 or any range between 2 and 300 micrometers.

Biosensors of the invention are also capable of detecting and quantifying the amount of a ligand that has bound to cell receptors by measuring the shift in reflected wavelength of light. For example, the wavelength shift at one or more distinct locations can be compared to positive and negative controls at other distinct locations to determine the amount of a ligand that has bound to cell receptors. Importantly, numerous such one or more distinct locations can be arranged on the biosensor surface, and the biosensor can comprise an internal surface of a vessel such as an about 2, 6, 8, 24, 48, 96, 384, 1536, 3456, 9600 or more well-microtiter plate. As an example, where 96 biosensors are attached to a holding fixture and each biosensor comprises about 100 distinct locations, about 9600 biochemical assays can be performed simultaneously.

All patents, patent applications, and other scientific or technical writings referred to anywhere herein are incorporated by reference in their entirety. The invention illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms, while retaining their ordinary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by embodiments, optional features, modification and variation of the concepts herein disclosed may be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

### EXAMPLES

### Example 1

HEK293 cells expressing type 1 angiotensin II receptors (AT1R) (60 k/well) were plated on a CA2 BIND™ colorimetric resonant reflectance biosensor microwell plate allowed to attach for 5 hours. Cells were then washed with starvation media, starved overnight, treated with ligands, and PWV responses were monitored for 70 minutes. The ligands were Ang II, which is a full agonist, or SII, which is a biased agonist. SII produced a PWV response that was smaller in magnitude and slower in onset as compared to the Ang II PWV response. See FIGS. 1A and 1B.

### Example 2

HEK293 cells expressing β-2 adrenergic receptors (β2AR) (60 k/well) were plated on a CA2 BIND™ colorimetric resonant reflectance biosensor microwell plate allowed to attach overnight. Cells were then washed with starvation media, starved for 5 hours, treated with ligands, and PWV responses were monitored for 70 minutes. The ligands were isoproterenol, which is a full agonist, or carvediolol, which is a biased agonist. Carvediolol produced a PWV response that was smaller in magnitude and slower in onset as compared to the isoproterenol PWV response. See FIGS. 2A and 2B.

### Example 3

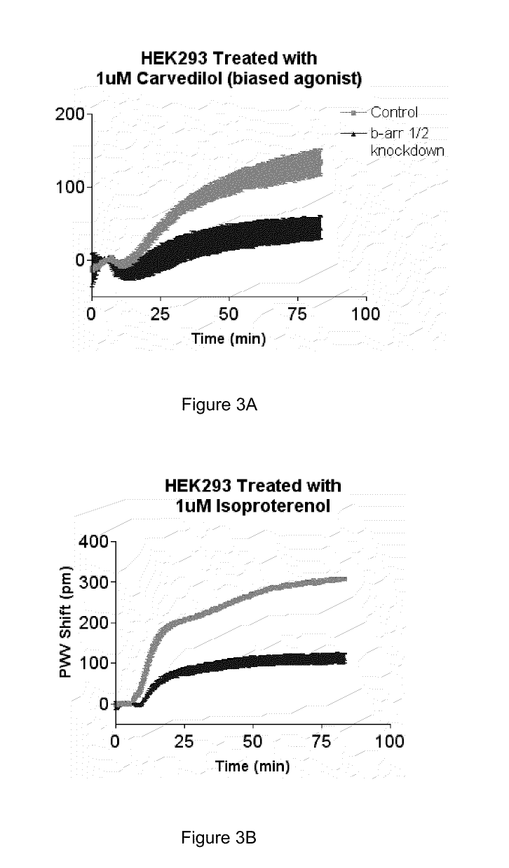

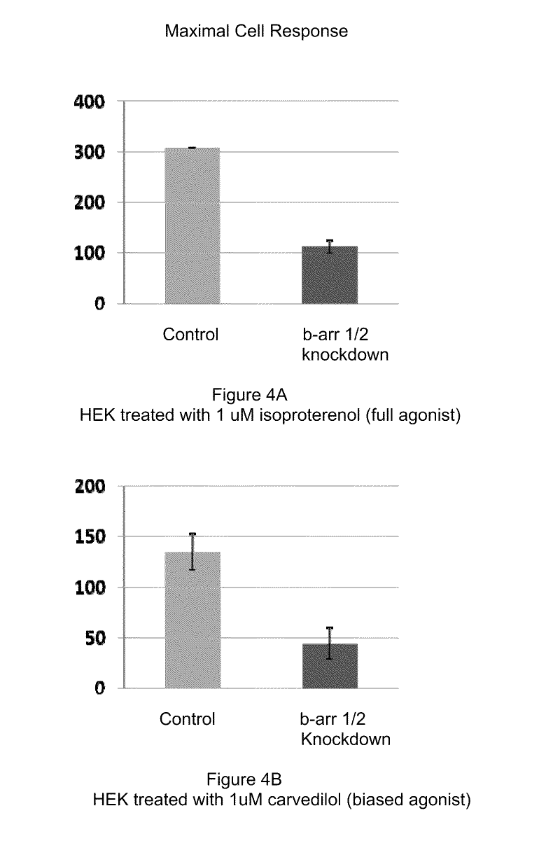

β-arrestins 1 and 2 are often activated by biased agonists, and are thought to be the primary signal trasnducer for biased agonists. β-arrestins 1 and 2 were knocked down with siRNA molecules in HEK293 cells expressing the β2 adrenergic receptor. Scrambled siRNA was used as control. Forty-eight hours after siRNA transfection, the cells were treated with either 1 uM isoproterenol (full agonist) or 1 uM carvedilol (biased agonist) and responses on a colorimetric resonant reflectance biosensor multiwall plate were monitored. All data are mean+/−SD of four replicate samples. Knockdown of β-arrestin decreased cell response to full and biased GPCR agonists. See FIGS. 3A-B and 4A-B. FIGS. 3A-B show the real time PWV responses over 80 minutes. The top lines are the controls treated with scrambled siRNA. The bottom lines are the experimental cells treated with siRNA molecules that are specific for β-arrestin 1 and 2 proteins. FIGS. 4A-B show the maximal cell response. The y axis is PWV shift (pm).

Knockdown of β-arrestin 1 and 2 proteins by siRNA molecules decreased the PWV signal in response to both full and biased agonists, demonstrating that signaling through biased agonists can be detected using the methods of the invention.

We claim:

1. A method of determining if a ligand or stimulus is a biased agonist comprising:
a) immobilizing or placing one or more cells on a surface of a colorimetric resonant reflectance optical biosensor;
b) applying a ligand or stimulus to the one or more cells;
c) detecting colorimetric resonant reflectance optical peak wavelength values (PWVs) for the one or more cells over time; and
d) comparing the PWVs of step (c) to PWVs over time of a full agonist acting on the same or similar one or more cells of step (a);
wherein, the ligand or stimulus is a biased agonist if the PWVs of the ligand or stimulus over time are smaller in magnitude or later in onset or both than the PWVs over time of the full agonist.

2. The method of claim 1, wherein no detection labels are associated with the cells, ligand and stimulus.

3. The method of claim 1, wherein the PWVs are detected for five or more minutes.

4. The method of claim 1, wherein the one or more cells are immobilized to the surface of the colorimetric resonant reflectance optical biosensor by one or more specific binding substances.

5. The method of claim 4, wherein the one or more specific binding substances are one or more extracellular matrix ligands.

6. A method of determining if a ligand or stimulus is a not a full agonist comprising:
a) immobilizing or placing one or more cells on a surface of a colorimetric resonant reflectance optical biosensor;
b) applying a ligand or stimulus to the one or more cells;
c) detecting colorimetric resonant reflectance optical peak wavelength values (PWVs) for the one or more cells over time; and
d) comparing the PWVs of step (c) to PWVs over time of a full agonist acting on the same or similar one or more cells of step (a);
wherein, the ligand or stimulus is not a full agonist if the PWVs of the ligand or stimulus over time are smaller in magnitude or later in onset or both than the PWVs over time of the full agonist.

7. The method of claim 6, wherein no detection labels are associated with the cells, ligand and stimulus.

8. The method of claim 6, wherein the PWVs are detected for five or more minutes.

9. The method of claim 6, wherein the one or more cells are immobilized to the surface of the colorimetric resonant reflectance optical biosensor by one or more specific binding substances.

10. The method of claim 9, wherein the one or more specific binding substances are one or more extracellular matrix ligands.

11. A method of determining if a ligand or stimulus is a biased agonist comprising:
a) immobilizing or placing one or more cells on a surface of a grating based waveguide biosensor;
b) applying a ligand or stimulus to the one or more cells;
c) detecting changes in effective refractive indices for the one or more cells over time; and
d) comparing the effective refractive indices of step (c) to effective refractive indices over time of a full agonist acting on the same or similar one or more cells of step (a);
wherein, the ligand or stimulus is a biased agonist if the effective refractive indices of the ligand or stimulus over time are smaller in magnitude or later in onset or both than the effective refractive indices over time of the full agonist.

12. The method of claim 11, wherein no detection labels are associated with the cells, ligand and stimulus.

13. The method of claim 11, wherein the effective refractive indices are detected for five or more minutes.

14. The method of claim 11, wherein the one or more cells are immobilized to the surface of the grating based waveguide biosensor by one or more specific binding substances.

15. The method of claim 14, wherein the one or more specific binding substances are one or more extracellular matrix ligands.

16. A method of determining if a ligand or stimulus is a not a full agonist comprising:
a) immobilizing or placing one or more cells on a surface of a grating based waveguide biosensor;
b) applying a ligand or stimulus to the one or more cells;
c) detecting changes in effective refractive indices for the one or more cells over time; and
d) comparing the effective refractive indices of step (c) to effective refractive indices over time of a full agonist acting on the same or similar one or more cells of step (a);
wherein, the ligand or stimulus is not a full agonist if the effective refractive indices of the ligand or stimulus over time are smaller in magnitude or later in onset or both than the effective refractive indices over time of the full agonist.

17. The method of claim 16, wherein no detection labels are associated with the cells, ligand and stimulus.

18. The method of claim 16, wherein the changes in effective refractive indices are detected for five or more minutes.

19. The method of claim 16, wherein the one or more cells are immobilized to the surface of a grating based waveguide biosensor by one or more specific binding substances.

20. The method of claim 19, wherein the one or more specific binding substances are one or more extracellular matrix ligands.
